(19) United States
(12) Patent Application Publication
(10) Pub. No.: US 2013/0344153 A1
(43) Pub. Date: Dec. 26, 2013

(54) METHOD FOR REDUCING INCIDENCE OR RATE OF DEVELOPMENT OF SKIN CANCERS AND RELATED CONDITIONS

(71) Applicant: Clinuvel Pharmaceuticals Limited, Melbourne (AU)

(72) Inventor: Philippe Wolgen, Melbourne (AU)

(21) Appl. No.: 14/016,828

(22) Filed: Sep. 3, 2013

Related U.S. Application Data
(63) Continuation of application No. 12/438,987, filed on Dec. 20, 2009.

(30) Foreign Application Priority Data
Aug. 28, 2006 (AU) 2006904672

(51) Int. Cl. A61K 38/22

(52) U.S. Cl. CPC A61K 38/22
USPC 424/489; 514/10.7; 424/400

(57) ABSTRACT

A method for treatment to reduce the incidence or rate of development of skin cancers and related conditions caused by or exacerbated by or associated with UVR-induced skin damage in an immuno-compromised subject, such as an organ transplant patient, comprises the step of administering to said subject an amount of an alpha-MSH analogue effective to protect the skin of the subject from UVR-induced skin damage.

### FIELD OF THE INVENTION

The present invention relates broadly to a method for reducing the incidence or rate of development of skin cancers and related conditions that are caused by ultraviolet radiation (UVR)-induced skin damage in immuno-compromised and immune-deficient patients.

### BACKGROUND

Alpha melanocyte stimulating hormone (alpha-MSH) is released from UVR exposed melanocytes and keratinocytes in human skin following exposure to ultraviolet radiation. It is understood to act on the melanocortin-1-receptors (MC1R) to, exclusively in melanocytes, induce synthesis of the brownish-black melanin pigment. MC1R are expressed on keratinocytes as well as number of other cells including, but not exclusively, immunological cells such as dendritic/Langerhans cells, neutrophils, microglia and monocytes as well as astrocytes, and endothelial cells.

It has previously been disclosed that a super-potent derivative of alpha-MSH, Nle⁴-D-Phe⁷-α-MSH, can induce melanin synthesis in human volunteers. Nle⁴-D-Phe⁷-α-MSH contains two amino acid substitutions and is approximately 10 to 1,000-fold more potent than the native hormone at inducing pigmentation in experimental systems such as the frog skin bioassay or in cultured human keratinocytes. It has been postulated that increasing melanin alone, whether through exposure to UVR or by chemical agents, can confer an increased level of photoprotection. However, increased levels of melanin in black- or dark-skinned individuals does not abrogate the risk of skin cancer and only minimally protects the skin from further damage.

It is known that UVR exposure damages DNA and promotes the development of skin cancer. High rates of skin cancers and related conditions in immuno-compromised patients are a significant clinical problem.

Accordingly, there is a need for methods for reducing the incidence or rate of development of skin cancers and related conditions that are caused or exacerbated by or associated with UVR-induced skin damage in an immuno-compromised subject, particularly a human subject.

The present invention provides a method for reducing the incidence or rate of development of skin cancers and related conditions in an immuno-compromised subject that are caused by UVR-induced skin damage by administration of an alpha-MSH analogue.

Bibliographic details of the publications referred to in this specification by reference number are collected at the end of the specification.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Those skilled in the art will appreciate that the invention described herein is susceptible to variations and modifications other than those specifically described. It is to be understood that the invention includes all such variations and modifications, the invention also includes all of the steps, features, compositions and compounds referred to or indicated in this specification, individually or collectively, and any and all combinations or any two or more of said steps or features.

### SUMMARY OF THE INVENTION

In one aspect, the present invention provides a method of treatment to reduce the incidence or rate of development of skin cancers and related conditions caused by or exacerbated by or associated with UVR-induced skin damage in an immuno-compromised subject, particularly a human subject, which comprises the step of administering to said subject an amount of an alpha-MSH analogue effective to protect the skin of the subject from UVR-induced skin damage.

In another aspect, the present invention provides the use of an alpha-MSH analogue in, or in the manufacture of a medicament for, treatment to reduce the incidence or rate of development of skin cancers and related conditions caused or exacerbated by or associated with UVR-induced skin damage in an immuno-compromised subject, particularly a human subject.

In yet another aspect, the invention provides an agent for use in treatment to reduce the incidence of or rate of development of skin cancers and related conditions caused by or exacerbated by or associated with UVR-induced skin damage in an immuno-compromised subject, particularly a human subject, comprising an alpha-MSH analogue.

### DETAILED DESCRIPTION OF THE INVENTION

Before the present methods are disclosed and described, it is to be understood that the aspects described below are not limited to specific methods or uses as such may, of course, vary. It is also to be understood that the terminology used herein is for the purpose of describing particular aspects only and is not intended to be limiting.

In this specification and in the claims that follow, reference will be made to a number of terms that shall be defined to have the following meanings:

Throughout this specification, unless the context requires otherwise, the word “comprise,” or variations such as “comprises” or “comprising,” will be understood to imply the inclusion of a stated integer or step or group of integers or steps but not the exclusion of any other integer or step or group of integers or steps.

It must be noted that, as used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

“Optional” or “optionally” means that the subsequently described event or circumstance can or cannot occur, and that the description includes instances where the event or circumstance occurs and instances where it does not.

Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

References in the specification and concluding claims to parts by weight, of a particular element or component in a composition or article, denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included.

By “contacting” is meant an instance of exposure by close physical contact of at least one substance to another substance. For example, contacting can include contacting a substance, such as a pharmacologic agent, with a cell. A cell can be contacted with a test compound, for example, an analogue of alpha-MSH, by adding the agent to the culture medium (by continuous infusion, by bolus delivery, or by changing the medium to a medium that contains the agent) or by adding the agent to the extracellular fluid in vivo (by local delivery, systemic delivery, intravenous injection, bolus delivery, or continuous infusion). The duration of contact with a cell or group of cells is determined by the time the test compound is present at physiologically effective levels or at presumed physiologically effective levels in the medium or extracellular fluid bathing the cell.

The term “immuno-compromised” means having an immune system that has been impaired by disease (such as AIDS) or treatment, particularly immune suppressive therapy. Thus, a subject which has an immune deficiency, that is, has an immune system in which the ability to fight infectious disease is compromised or entirely absent, is said to be immuno-compromised.

The terms “prophylactic treatment”, “prevention” or “preventing” mean the administration of an active compound or composition to a subject at risk for an undesirable condition. The condition can include a disease, disorder or reaction, or a predisposition to a disease, disorder or reaction. Prophylactic treatment can range from a reduction in the risk for the condition or of the severity of the condition to the complete prevention of the condition.

The terms “therapeutic treatment” and “treating” mean the administration of an active compound or composition to a subject having an undesirable condition such as a disease, disorder or reaction. Therapeutic treatment can range from reduction in the severity of the condition in the subject to the complete recovery of the subject from the condition.

By “reducing the incidence or rate of” development of is meant reducing the likelihood of occurrences of an undesirable condition (such as skin cancer or a related condition), as well as reducing the occurrences of the condition or reducing or slowing down the rate of development of the condition (both in number and in time), and reducing the severity of the occurrences of the condition.

By “effective amount and time” means an amount and time needed to achieve the desired result or results, e.g., preventing UVR-induced skin damage or further damage which leads to skin cancers or related conditions in a patient.

By “induce” means initiating or promoting a desired response or result that was not present prior to the induction step. The term “induce” also includes the term “potentiate.”

By “intermittent” means administering an active compound or composition in a series of discreet (constant or variable) doses over a determined period, e.g., a period of sustained release comprising of greater than 24 hours of an alpha-MSH analogue every two months.

The term “potentiate” means sustaining a desired response at the same level prior to the potentiating step or increasing the desired response over a period of time.

The term “melanogenesis” as referred to herein is defined as the ability of a subject to produce and release (into the dermal layers) melanins by melanin-producing cells, or melanocytes.

The term “epidermal tissue” as referred to herein includes in particular the skin of a subject.

Disclosed are compounds, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. This concept applies to all aspects of this disclosure including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Described herein are methods for treating an immuno-compromised patient to reduce the incidence or rate of development of skin cancers and related conditions caused or exacerbated by or associated with UVR-induced skin damage.

In one aspect, the invention provides a method of treatment to reduce the incidence or rate of development of skin cancers and related conditions caused or exacerbated by or associated with UVR-induced skin damage in an immuno-compromised subject, particularly a human subject, which comprises the step of administering to said subject an amount of an alpha-MSH analogue effective to protect the skin of the subject from UVR-induced skin damage.

Preferably, the alpha-MSH analogue is administered at a level not exceeding 100 ng/ml in the plasma of the subject for a period of at least 24 hours. Preferably also, the administration of the alpha-MSH analogue to the subject is systemic administration, even more preferably intermittent systemic administration.

Preferably, the subject is a human subject.

In another aspect, the present invention provides the use of an alpha-MSH analogue in, or in the manufacture of a medicament for, treatment to reduce the incidence or rate of development of skin cancers and related conditions caused or exacerbated by or associated with UVR-induced skin damage in an immuno-compromised subject, particularly a human subject.

The present invention is particularly directed at reducing the incidence or rate of development of melanoma skin cancers (MSC) and most particularly of nonmelanoma skin cancers (NMSC) in patients, including in particular squamous cell carcinomas (SCC) and basal cell carcinomas (BCC), as well as related conditions, in particular actinic keratosis (AK) (or solar keratosis) which is a premalignant condition of the skin which may often progress to SCC.

It has been shown that humans who are fair-skinned (e.g., having Type 1 or Type 2 skin on the Fitzpatrick scale as confirmed/ratified by the WHO, see Fitzpatrick, 1988—reference 3) and who are frequently exposed to UVR are most likely to develop AK. In addition, a particular subpopulation who are highly susceptible to AK and SCC has been identified, these are humans who are immuno-compromised (that is patients having suppressed systemic and local immune response/systems), particularly organ transplant patients, such as kidney, liver and heart transplant patients who are on immune suppressive therapy to prevent rejection of the transplanted organ, and others who are on prolonged immune suppressive therapy due to other life-threatening diseases. The incidence of recurring skin cancers in these transplant patients, and those who receive immune suppressive therapy, is a substantial clinical problem (see, for example, Euvrard et al., 2006—reference 2, and references 4 to 14 hereinafter).

Accordingly, in a particular embodiment of the invention, the human subject may be an immuno-compromised patient receiving immune suppressive therapy, particularly an organ transplant patient.

The term “alpha-MSH analogue” referred to herein is defined as a derivative of alpha-MSH which exhibits agonist activity for the melanocortin-1 receptor (MC1R), the receptor to which alpha-MSH binds to initiate the production of melanin within a melanocyte. Such derivatives include derivatives in which (i) one or more amino acid residues are deleted from the native alpha-MSH molecule at the N-terminal end, the C-terminal end, or both; and/or (ii) one or more amino acid residues of the native alpha-MSH molecule are replaced by another natural, non-natural or synthetic amino acid residue; and/or (iii) an intramolecular interaction forms as a cyclic derivative.

The use of any alpha-MSH analogue is contemplated in the methods described herein. Several derivatives of α-MSH have been synthesized. In one aspect, the alpha-MSH analogues described in U.S. Pat. Nos. 4,457,864, 4,485,039, 4,866,038, 4,918,055, 5,049,547, 5,674,839 and 5,714,576 and Australian Patents Nos. 597630 and 618733, which are herein incorporated by reference for their teachings with respect to alpha-MSH analogues and their synthesis thereof, can be used herein.

In one aspect, the alpha-MSH analogue may be a compound as disclosed in Australian Patent No. 597630, selected from:

(a) compounds of the formula:

(SEQ ID NO: 1)
Ac-Ser-Tyr-Ser-M-Gln-His-D-Phe-Arg-Trp-Gly-Lys-
Pro-Val-NH₂

wherein M is Met, Nle or Lys; and
(b) compounds of the formula:

| | |
|---|---|
| R1-W-X-Y-Z-R₂ | (SEQ ID NO: 2) |

wherein

### R₁ is Ac-Gly-, Ac-Met-Glu, Ac-Nle-Glu-, or Ac-Tyr-Glu-;

### W is -His- or -D-His-;

X is -Phe-, -D-Phe-, -Tyr-, -D-Tyr-, or -(pNO₂)D-Phe⁷-;

### Y is -Arg- or -D-Arg-;

### Z is -Trp- or -D-Trp-; and

### R₂ is —NH₂; -Gly-NH₂; or -Gly-Lys-NH₂.

In another aspect, the alpha-MSH analogue may be selected from cyclic analogues which are disclosed in Australian Patent No. 618733 where an intramolecular interaction (such as a disulfide or other covalent bond) exists (1) between the amino acid residue at position 4 and an amino acid residue at position 10 or 11, and/or (2) between the amino acid residue at position 5 and the amino acid residue at position 10 or 11.

The alpha-MSH analogue may be a linear analogue as disclosed in U.S. Pat. No. 5,674,839 selected from the group consisting of

(SEQ ID NO: 3)
Ac-Ser-Tyr-Ser-Nle-Glu-His-D-Phe-Arg-Trp-Lys-Gly-Pro-Val-NH₂
(SEQ ID NO: 4)
Ac-Ser-Tyr-Ser-Nle-Asp-His-D-Phe-Arg-Trp-Lys-Gly-Pro-Val-NH₂
(SEQ ID NO: 5)
Ac-Nle-Glu-His-D-Phe-Arg-Trp-Lys-Gly-Pro-Val-NH₂
(SEQ ID NO: 6)
Ac-Nle-Asp-His-D-Phe-Arg-Trp-Lys-Gly-Pro-Val-NH₂
(SEQ ID NO: 7)
Ac-Nle-Asp-His-D-Phe-Arg-Trp-Gly-NH₂
(SEQ ID NO: 8)
Ac-Nle-Glu-His-D-Phe-Arg-Trp-Lys-NH₂
(SEQ ID NO: 9)
Ac-Nle-Asp-His-D-Phe-Arg-Trp-Lys-NH₂
(SEQ ID NO: 10)
Ac-Nle-Glu-His-D-Phe-Arg-Trp-Orn-NH₂
(SEQ ID NO: 11)
Ac-Nle-Asp-His-D-Phe-Arg-Trp-Orn-NH₂
(SEQ ID NO: 12)
Ac-Nle-Glu-His-D-Phe-Arg-Trp-Dab-NH₂
(SEQ ID NO: 13)
Ac-Nle-Asp-His-D-Phe-Arg-Trp-Dab-NH₂
(SEQ ID NO: 14
Ac-Nle-Glu-His-D-Phe-Arg-Trp-Dpr-NH₂
(SEQ ID NO: 15)
Ac-Nle-Glu-His-Phe-Arg-Trp-Lys-NH₂
(SEQ ID NO: 16)
Ac-Nle-Asp-His-Phe-Arg-Trp-Lys-NH₂

The alpha-MSH analogue may also be a cyclic analogue as disclosed in U.S. P. No. 5,674,839, selected from the group consisting of:

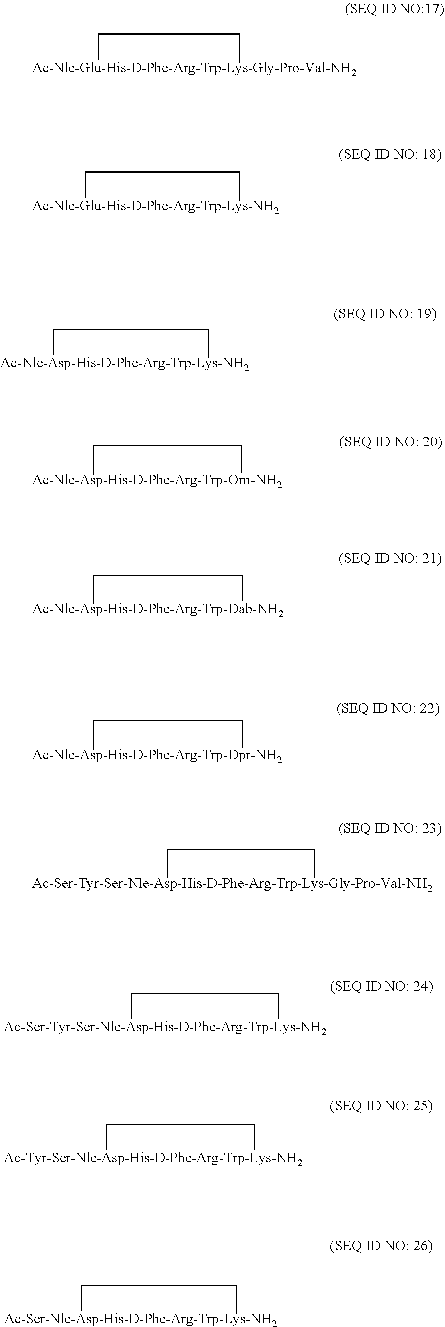

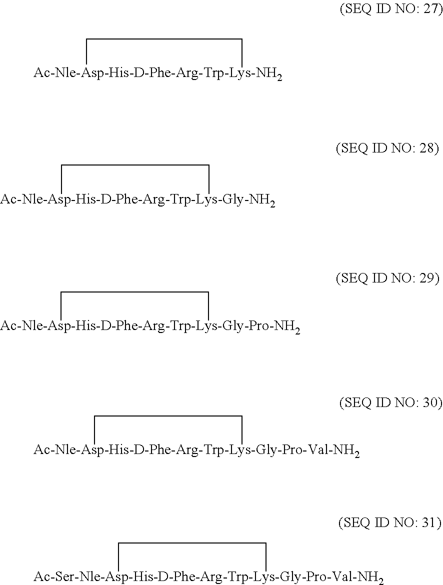

Where referred to herein, Ala=alanine, Arg=arginine, Dab=2,4-diaminobutyric acid, Dpr=2,3-diaminopropionic acid, Glu=glutamic acid, Gly=glycine, His=histidine, Lys=lysine, Met=methionine, Nle=norleucine, Orn=ornithine, Phe=phenylalanine, (pNO₂)Phe=paranitrophenylalanine, Plg=phenylglycine, Pro=proline, Ser=serine, Trp=tryptophan, TrpFor=N¹⁻ formyl-tryptophan, Tyr=tyrosine, Val=valine. All peptides are written with the acyl-terminal end at the left and the amino terminal end to the right; the prefix “D” before an amino acid designates the D-isomer configuration, and unless specifically designated otherwise, all amino acids are in the L-isomer configuration. In one aspect, the alpha-MSH analogue can be

[D-Phe⁷]-alpha-MSH,
[Nle⁴, D-Phe⁷]-alpha-MSH,
[D-Ser¹, D-Phe⁷]-alpha-MSH,
[D-Tyr², D-Phe⁷]-alpha-MSH,
[D-Ser³, D-Phe⁷]-alpha-MSH,
[D-Met⁴, D-Phe⁷]-alpha-MSH,
[D-Glu⁵, D-Phe⁷]-alpha-MSH,
[D-His⁶, D-Phe⁷]-alpha-MSH,
[D-Phe⁷, D-Arg⁸]-alpha-MSH,
[D-Phe⁷, D-Trp⁹]-alpha-MSH,
[D-Phe⁷, D-Lys¹¹]-alpha-MSH,
[D-Phe-⁷, D-Pro¹²]-alpha-MSH,
[D-Phe⁷, D-Val¹³]-alpha-MSH,
[D-Ser¹, Nle⁴, D-Phe⁷]-alpha-MSH,
[D-Tyr², Nle⁴, D-Phe⁷]-alpha-MSH,
[D-Ser³, Nle⁴, D-Phe⁷]-alpha-MSH,
[Nle⁴, D-Glu⁵, D-Phe⁷]-alpha-MSH,
[Nle⁴, D-His⁶, D-Phe⁷]-alpha-MSH,
[Nle⁴, D-Phe⁷, D-Arg⁸]-alpha-MSH,
[Nle⁴, D-Phe⁷, D-Trp⁹]-alpha-MSH,
[Nle⁴, D-Phe⁷, D-Lys¹¹]-alpha-MSH,
[Nle⁴, D-Phe⁷, D-Pro¹²]-alpha-MSH,
[Nle⁴, D-Phe⁷, D-Val¹³]-alpha-MSH,

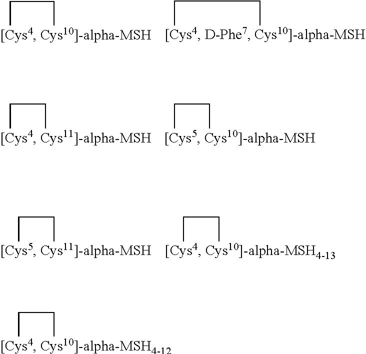

[Nle⁴, D-Phe⁷]-alpha-MSH₄₋₁₀,
[Nle⁴, D-Phe⁷]-alpha-MSH₄₋₁₁,
[D-Phe⁷]-alpha-MSH₅₋₁₁,
[Nle⁴, D-Tyr⁷]-alpha-MSH₄₋₁₁,
[(pNO₂)D-Phe⁷]-alpha-MSH₄₋₁₁,
[Tyr⁴, D-Phe⁷]-alpha-MSH₄₋₁₀,
[Tyr⁴, D-Phe⁷]-alpha-MSH₄₋₁₁,
[Nle⁴]-alpha-MSH₄₋₁₁,
[Nle⁴, (pNO₂)D-Phe⁷]-alpha-MSH₄₋₁₁,
[Nle⁴, D-His⁶]-alpha-MSH₄₋₁₁,
[Nle⁴, D-His⁶, D-Phe⁷]-alpha-MSH₄₋₁₁,
[Nle⁴, D-Arg⁸]-alpha-MSH₄₋₁₁,
[Nle⁴, D-Trp⁹]-alpha-MSH₄₋₁₁,
[Nle⁴, D-Phe⁷, D-Trp⁹]-alpha-MSH₄₋₁₁,
[Nle⁴, D-Phe⁷]-alpha-MSH₄₋₉, or
[Nle⁴, D-Phe⁷, D-Trp⁹]-alpha-MSH₄₋₉.

In a further aspect, the alpha-MSH analogue is

[Nle⁴, D-Phe⁷]-alpha-MSH₄₋₁₀,
[Nle⁴, D-Phe⁷]-alpha-MSH₄₋₁₁,
[Nle⁴, D-Phe⁷, D-Trp⁹]-alpha-MSH₄₋₁₁, or
[Nle⁴, D-Phe⁷]-alpha-MSH₄₋₉.

In a particularly preferred aspect, the alpha-MSH analogue is [Nle⁴, D-Phe⁷]-alpha-MSH. In another aspect, as described above the alpha-MSH analogue may be a truncated derivative of alpha-MSH, including a truncated derivative in which one or more amino acid residues of the truncated native alpha-MSH molecule are replaced by another natural, non-natural or synthetic amino acid residue. Thus, the alpha-MSH analogue may be a truncated derivative such as the tetrapeptide alpha-MSH analogues of the formula:

| | |
|---|---|
| R3-His-D-Phe-Arg-Trp-NH₂ | (SEQ ID NO: 32) |

wherein R₃ is Ac, n-pentadecanoyl, or 4-phenylbutyryl;
as disclosed by Abdel-Malek et al., 2006—reference 15.

The alpha-MSH analogue may be administered in a sustained-release delivery system as disclosed in International Patent Application No. PCT/AU2005/000181 (WO 2006/012667), or topically using a transdermal delivery system as disclosed in International Patent Application No. PCT/AU2005/001552 (WO 2006/037188).

In treatment of an immuno-compromised subject in accordance with the present invention, the alpha-MSH analogue may be administered to the subject in association with immune suppression medication.

It will be appreciated that the actual preferred amounts of the alpha-MSH analogue in a specified case will vary according to the specific compounds being utilized, the particular compositions formulated, the mode of application, and the particular situs and subject being treated. Dosages for a given host can be determined using conventional considerations, e.g., by customary comparison of the differential activities of the subject compounds and of a known agent, e.g., by means of an appropriate conventional pharmacological protocol. Physicians and formulators, skilled in the art of determining doses of pharmaceutical compounds, will have no problems determining doses for prophylactically treating a patient to reduce the incidence of skin cancers and related conditions in the patient by administration of an amount of an alpha-MSH analogue by the methods described herein. In one aspect, the alpha-MSH analogue is administered in an amount which is effective to prophylactically treat the patient to reduce the incidence of skin cancers and related conditions in the patient.

Any of the alpha-MSH analogues useful herein can be administered to a subject using a variety of administration or delivery techniques known in the art. It is desirable to maintain low concentrations of the alpha-MSH analogue in the plasma of the subject to induce prophylactic treatment to reduce the incidence of skin cancers and related conditions in the subject. Therefore, the mode of administration will depend upon the subject to be treated and the alpha-MSH analogue selected. In various aspects, the alpha-MSH analogues can be administered orally or parenterally. The term “oral” is used herein to encompass administration of the compounds via the digestive tract. The term “parenteral” is used herein to encompass any route of administration, other than oral administration, by which the alpha-MSH analogue is introduced into the systemic circulation which includes, but is not limited to, intravenous, intramuscular, subcutaneous, intraperitoneal, intradermal, ocular, inhalable, rectal, vaginal, transdermal, topical, buccal, sublingual, or mucosal administration. The term “mucosal” as used herein encompasses the administration of the compounds by methods that employ the mucosa (mucous membranes) of the human body such as, but not limited to, buccal, intranasal, gingival, vaginal, sublingual, pulmonary, or rectal tissue. The term “transdermal” as used herein encompasses the administration of the compounds that go into the skin or go through the skin using formulations such as, but not limited to, transdermal formulations, buccal patches, skin patches, or transdermal patches. The term “topical” as used herein encompasses administration by applying conventional topical preparations such as creams, gels, or solutions for localized percutaneous delivery and/or by solution for systemic and/or localized delivery to areas such as, but not limited to the eye, skin, rectum, and vagina.

In one aspect, delivery systems composed of devices or compositions containing an alpha-MSH analogue can be manufactured that allow for the controlled-release, extended-release, modified-release, sustained-release, pulsatile-release, or programmed-release delivery of the alpha-MSH analogue in order to maintain concentration of the alpha-MSH analogue in the plasma of the subject. Depending on the delivery system or composition of a formulation or route of administration chosen, drugs or active pharmaceutical ingredients can be delivered for hours, weeks, or months following a single administration. Drug-delivery devices include, but are not limited to pumps, needle-free injectors, metered-dose inhalers, and the like. Transdermal compositions with or without penetration enhancers include but are not limited to transdermal patches, microneedles, and transdermal formulations that achieve drug delivery using inotophoresis, sonophoresis, electroporation, thermoporation, perfusion, adsorption and absorption. Other delivery systems include, but are not limited to, biodegradable or non-biodegradable rods or other shaped implants, fibers, microparticles, microspheres, microcapsules, nanospheres, nanocapsules, porous silicon nanoparticles, in situ gelling formulations, in situ bolus forming compositions, quick dissolving tablets and the like, buccal patches, films, tablets, capsules, osmotic pressure driven formulations, liquid filled capsules, liposomes and other lipid based compositions and the like, pegalation and the like, hydrogel formulations, emulsions, microemulsions, and suspensions.

In one aspect, polymeric delivery systems can be microparticles including, but not limited to microspheres, microcapsules, nanospheres and nanoparticles comprising biodegradable polymeric excipients, non-biodegradable polymeric excipients, or mixtures of polymeric excipients thereof, or the polymeric delivery systems can be, but not limited to rods or other various shaped implants, wafers, fibers, films, in situ forming boluses and the like comprising biodegradable polymeric excipients, non-biodegradable polymeric excipients, or mixtures thereof. These systems can be made from a single polymeric excipient or a mixture or blend of two or more polymeric excipients.

A suitable polymeric excipient includes, but is not limited to, a poly(diene) such as poly(butadiene) and the like; a poly(alkene) such as polyethylene, polypropylene, and the like; a poly(acrylic) such as poly(acrylic acid) and the like; a poly(methacrylic) such as poly(methyl methacrylate), a poly(hydroxyethyl methacrylate), and the like; a poly(vinyl ether); a poly(vinyl alcohol); a poly(vinyl ketone); a poly(vinyl halide) such as poly(vinyl chloride) and the like; a poly(vinyl nitrile), a poly(vinyl ester) such as poly(vinyl acetate) and the like; a poly(vinyl pyridine) such as poly(2-vinyl pyridine), poly(5-methyl-2-vinyl pyridine) and the like; a poly(styrene); a poly(carbonate); a poly(ester); a poly(orthoester) including a copolymer; a poly(esteramide); a poly(anhydride); a poly(urethane); a poly(amide); a cellulose ether such as methyl cellulose, hydroxyethyl cellulose, hydroxypropyl methyl cellulose, and the like; a cellulose ester such as cellulose acetate, cellulose acetate phthalate, cellulose acetate butyrate, and the like; a poly(saccharide), a protein, gelatin, starch, gum, a resin, and the like. These materials may be used alone, as physical mixtures (blends), or as co-polymers. Derivatives of any of the polymers listed above are also contemplated.

In one aspect, the polymeric excipient of the delivery system includes a biocompatible, non-biodegradable polymer such as, for example, a silicone, a polyacrylate; a polymer of ethylene-vinyl acetate; an acyl substituted cellulose acetate; a non-degradable polyurethane; a polystyrene; a polyvinyl chloride; a polyvinyl fluoride; a poly(vinyl imidazole); a chlorosulphonate polyolefin; a polyethylene oxide; or a blend or copolymer thereof.

In another aspect, the polymeric excipient includes a biocompatible, biodegradable polymer such as, for example, a poly(lactide); a poly(glycolide); a poly(lactide-co-glycolide); a poly(lactic acid); a poly(glycolic acid); a poly(lactic acid-co-glycolic acid); a poly(caprolactone); a poly(orthoester); a poly(phosphazene); a poly(hydroxybutyrate) or a copolymer containing a poly(hydroxybutarate); a poly(lactide-co-caprolactone); a polycarbonate; a polyesteramide; a polyanhydride; a poly(dioxanone); a poly(alkylene alkylate); a copolymer of polyethylene glycol and a polyorthoester; a biodegradable polyurethane; a poly(amino acid); a polyetherester; a polyacetal; a polycyanoacrylate; a poly(oxyethylene)/poly(oxypropylene)copolymer, or a blend or copolymer thereof.

In one aspect, the delivery system comprises an implant or rod, wherein the implant or rod comprises a biodegradable polymer, wherein the alpha-MSH analogue is imbedded within the implant or rod. In one aspect, the alpha-MSH analogue is encapsulated in an implant or rod composed of poly(lactide-co-glycolide), poly(lactide), poly(glycolide), or a mixture thereof. Lactide/glycolide polymers for drug-delivery formulations are typically made by melt polymerization through the ring opening of lactide and glycolide monomers. Some polymers are available with or without carboxylic acid end groups. When the end group of the poly(lactide-co-glycolide), poly(lactide), or poly(glycolide) is not a carboxylic acid, for example, an ester, then the resultant polymer is referred to herein as blocked or capped. The unblocked polymer, conversely, has a terminal carboxylic group. In one aspect, linear lactide/glycolide polymers are used; however star polymers can be used as well. In certain aspects, high molecular weight polymers can be used for medical devices, for example, to meet strength requirements. In other aspects, low molecular weight polymers can be used for drug-delivery and vaccine delivery products where resorption time and not material strength is as important. The lactide portion of the polymer has an asymmetric carbon. Commercially racemic DL-, L-, and D-polymers are available. The L-polymers are more crystalline and resorb slower than DL-polymers. In addition to copolymers comprising glycolide and DL-lactide or L-lactide, copolymers of L-lactide and DL-lactide are available. Additionally, homopolymers of lactide or glycolide are available.

In the case when the biodegradable polymer is poly(lactide-co-glycolide), poly(lactide), or poly(glycolide), the amount of lactide and glycolide in the polymer can vary. In one aspect, the biodegradable polymer contains 0 to 100 mole %, 40 to 100 mole %, 50 to 100 mole %, 60 to 100 mole %, 70 to 100 mole %, or 80 to 100 mole % lactide and from 0 to 100 mole %, 0 to 60 mole %, 10 to 40 mole %, 20 to 40 mole %, or 30 to 40 mole % glycolide, wherein the amount of lactide and glycolide is 100 mole %. In one aspect, the biodegradable polymer can be poly(lactide), 85:15 poly(lactide-co-glycolide), 75:25 poly(lactide-co-glycolide), or 65:35 poly(lactide-co-glycolide) where the ratios are mole ratios.

In one aspect, when the biodegradable polymer is poly(lactide-co-glycolide), poly(lactide), or poly(glycolide), the polymer has an intrinsic viscosity of from 0.15 to 1.5 dL/g, 0.25 to 1.5 dL/g, 0.25 to 1.0 dL/g, 0.25 to 0.8 dL/g, 0.25 to 0.6 dL/g, or 0.25 to 0.4 dL/g as measured in chloroform at a concentration of 0.5 g/dL at 30° C.

The amount of alpha-MSH analogue that is encapsulated or incorporated in the biodegradable polymer will vary depending upon the selection of the biodegradable polymer, the encapsulation or incorporation technique, and the amount of alpha-MSH to be delivered to the subject. In one aspect, the amount of alpha-MSH analogue encapsulated in the microcapsule, implant, or rod can be up to 50% by weight of the delivery system. In other aspects, the amount of alpha-MSH analogue encapsulated in the microcapsule, implant, or rod can be from 5 to 60, 10 to 50%, 15 to 40%, or 15 to 30% by weight of the delivery system.

In another aspect, where the alpha-MSH analogue is delivered by another delivery system such as a transdermal formulation, the amount of alpha-MSH analogue in the formulation can be from 0.001 to 10%, or 0.05 to 5% by weight of the formulation.

Other pharmaceutically-acceptable components can be encapsulated or incorporated in the delivery system in combination with the alpha-MSH analogue. For example, the pharmaceutically-acceptable component can include, but is not limited to, a fatty acid, a sugar, a salt, a water-soluble polymer such as polyethylene glycol, a protein, polysachamide, or carboxmethyl cellulose, a surfactant, a plasticizer, a high- or low-molecular-weight porosigen such as polymer or a salt or sugar, or a hydrophobic low-molecular-weight compound such as cholesterol or a wax. In another aspect, the delivery system comprises an implant or rod, wherein the alpha-MSH analogue is [Nle⁴, D-Phe⁷]-alpha-MSH in the amount from 15% to 45% by weight of the implant or rod, wherein the rod or implant comprises poly(lactide) or poly(lactide-co-glycolide) such as, for example, 85:15 poly(lactide-co-glycolide).

Any of the delivery systems described herein can be administered using techniques known in the art. In one aspect, the delivery system can be administered subcutaneously to the subject. In this aspect, the duration of administration can vary depending upon the amount of alpha-MSH analogue that is encapsulated and the biodegradable polymer selected. In one aspect, the delivery system is administered subcutaneously to the subject and releases the alpha-MSH analogue for a period of at least 2, 4, 6, 8, 10 or 12 days. In one aspect, the delivery system releases the alpha-MSH analogue in the subject for up to three months. In various other aspects, the delivery system releases the alpha-MSH analogue in the subject for 10 days, 15 days, 20 days, 25 days, or 30 days.

In one aspect, any of the alpha-MSH analogues can be combined with at least one pharmaceutically-acceptable carrier to produce a pharmaceutical composition. The pharmaceutical compositions can be prepared using techniques known in the art. In one aspect, the composition is prepared by admixing the alpha-MSH analogue with a pharmaceutically-acceptable carrier. The term “admixing” is defined as mixing the two components together so that there is no chemical reaction or physical interaction. The term “admixing” also includes the chemical reaction or physical interaction between the alpha-MSH analogue and the pharmaceutically-acceptable carrier.

Pharmaceutically-acceptable carriers are known to those skilled in the art. These most typically would be standard carriers for administration to humans, including solutions such as sterile water, saline, and buffered solutions at physiological pH.

Molecules intended for pharmaceutical delivery may be formulated in a pharmaceutical composition. Pharmaceutical compositions may include carriers, thickeners, diluents, buffers, preservatives, surface active agents and the like in addition to the molecule of choice. Pharmaceutical compositions may also include one or more active ingredients such as antimicrobial agents, anti-inflammatory agents, anesthetics, and the like.

Preparations for administration include sterile aqueous or non-aqueous solutions, suspensions, and emulsions. Examples of non-aqueous carriers include water, alcoholic/aqueous solutions, emulsions or suspensions, including saline and buffered media. Parenteral vehicles, if needed for collateral use of the disclosed compositions and methods, include sodium chloride solution, Ringer's dextrose, dextrose and sodium chloride, lactated Ringer's, or fixed oils. Intravenous vehicles, if needed for collateral use of the disclosed compositions and methods, include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, ointments, suppositories, sprays, liquids and powders. Conventional pharmaceutical carriers, aqueous, powder or oily bases, thickeners and the like may be necessary or desirable. The alpha-MSH analogue can be admixed under sterile conditions with a physiologically acceptable carrier and any preservatives, buffers, propellants, or absorption enhancers as may be required or desired. Reference is made to documents cited herein, e.g., U.S. Pat. No. 5,990,091, WO 98/00166, and WO 99/60164, for the preparation of compositions for topical applications, e.g., viscous compositions that can be creams or ointments, as well as compositions for nasal and mucosal administration.

In the case when the composition is administered mucosally, ocularly, intranasally, or by inhalation, the formulation can be in the form of a drop, a spray, an aerosol, or a sustained release format. The spray and the aerosol can be achieved through use of the appropriate dispenser. The sustained release format can be an ocular insert, erodible microparticulates, swelling mucoadhesive particulates, pH sensitive microparticulates, nanoparticles/latex systems, ion-exchange resins and other polymeric gels and implants (Ocusert, Alza Corp., California; Joshi, A., S. Ping and K. J. Himmelstein, Patent Application WO 91/19481). These systems maintain prolonged drug contact with the absorptive surface preventing washout and nonproductive drug loss.

The invention is further described with reference to the following non-limiting Examples which illustrate various embodiments of the invention.

### Example 1

Hairless mouse strains such as the HRA-Skh-1 mice are a standard mouse model used to study solar damage to human skin (Canfield et al., 1985—reference 1). Exposure of the hairless mouse to UV light mimics “sunburn” in humans. With continued irradiation treatment, this on-going damage is reflected in progressive thickening of the skin which histologically mimics hyperkeratinization and elastosis associated with photoaging and chronically sun-exposed skin in humans. Pre-malignant tumours begin to appear within several weeks of completion of the ultra violet light regimen. Over an ensuing time period there is a progressive development of pre-malignant and malignant tumours, the histology and behaviour of which closely mimic keratoses and pre-malignant and malignant skin cancers that develop in humans in response to sunlight.

Hairless albino Sh:HR-1 mice are divided into two groups each containing 20 animals. The first group receiving Nle⁴-D-Phe⁷-alpha-MSH, and the second group being a control group. Both groups receive one minimal erythemal dose of UVB light to stimulate the toxic effect of sunlight on the skin daily for 10 weeks. At weeks 11, 13, 14, 15, 16 and 17 the number of skin tumours on each animal is measured and calculated as tumour multiplicity, i.e. average number of tumours per mouse. Protection against UVB induced carcinogenesis is observed.

### Example 2

Patients who have been eligible to receive a donor organ, are administered immune-suppressive medication post-transplant surgery for a prolonged amount of time, most often for the remainder of the life of patient. Examples of the immune suppressive medication, and any combination thereof are: Corticosteroids; Azathioprine; Cyclosporine; Mycophenolate mofetil; Tacrolimus; and Sirolimus.

The prolonged administration of the aforementioned medication, in any possible combination, results in immune suppression of the subject. The dose of the immunosuppressant medication(s) as defined by the administration per mg/kg/day per subject varies clinically. Furthermore, it is clinically observed that fair-skinned patients (Fitzpatrick I and II skin types) receiving prolonged immunosuppressants are prone to developing UV-related dermal lesions such as actinic keratosis, keratoacanthosis, basal cell carcinoma, squamous cell carcinoma and melanoma. It is clinically seen that 2 to 5 years post-transplantation, i.e. after receiving a donor graft or organ, the patient is most ‘sensitive’ or susceptible to develop any of the UV-related skin disorders.

Concomitant therapy with an alpha-MSH analogue as provided by the present invention, reduces the incidence or rate of development of actinic keratosis, keratoacanthosis, basal cell carcinoma, squamous cell carcinoma and melanoma in this subpopulation of immuno-compromised patients.

A double-blind, randomised, placebo-controlled study is carried out in immune suppressed organ transplant patients with actinic keratoses on their facial region, scalp and extremities. The study is conducted in accordance with the Declaration of Helsinki and its revisions, ICH guidelines for Good Clinical Practice (GCP) governing the conduct of studies, and all applicable local regulations.

Subjects are recruited from a database of organ transplant patients. According to the main criteria for entry into the study, eligible subjects are male or female organ transplant recipients (aged 18-75 years) with stable transplant function who received their transplant at least six months prior to study entry, and who have at least one biopsy-positive AK or SCC. Written informed consent to the performance of all study specific procedures is obtained from each patient. Subjects undergo screening/baseline evaluations to determine eligibility within seven days prior to the first administration of study drug. Patients are enrolled and randomised in equal numbers (ratio 1:1) to receive either the study drug (Nle⁴-D-Phe⁷-alpha-MSH) or placebo. All randomised subjects receive their first implant subcutaneously on Study Day 0, followed by additional doses on Days 60, 120, 180, 240 and 300.

All patients are given diaries to record daily exposure to sunlight, any adverse events experienced and concomitant medications. Participants visit the clinic on Days −7, 0, 15, 30, 60, 90, 120, 150, 180, 210, 240, 170, 300, 330 and 360 for the counting of skin lesions, or the measurement of skin melanin density. Safety is carefully monitored throughout the study. In addition, a home visit occurs 24 hours after each implant administration (six home visits) for a drug level and safety blood check (biochemistry).

The study drug is administered subcutaneously from slow-release implants containing 20 mg/study drug/implant contained in a poly(D,L-lactide) implant core, giving sustained release of 20 mg study drug over 10 to 15 days. The placebo implant contains only poly(D,L-lactide).

The criteria for evaluation of the study are efficacy analyses and safety analyses:

### Efficacy Analyses:

The primary efficacy analysis will compare the number of clinical AK lesions and the number of SCCs developing between Day 0 and Day 360 for patients in each of the treatment groups. These will be compared by an appropriate statistical method. Null hypothesis: there is no difference between treatment groups.

The secondary efficacy analyses will compare changes in melanin density from Day 0 to Day 360 in each of the treatment groups. These will be compared by an appropriate statistical method. Null hypothesis: there is no difference between treatment groups.

### Safety Analyses:

The number of participants with treatment-emergent adverse events will be summarised by MedDRA preferred term and body system for each treatment group. Treatment-emergent events will be further summarised by intensity, seriousness, outcome and relationship to study drug. The number of participants who prematurely terminate treatment due to adverse events related to study medication will be summarised. Clinical laboratory data will be summarised for each treatment group.

### REFERENCES

1. Canfield et al. (1985), Pathology, 17:613-616.
2. Euvrard, S. et al. Subsequent skin cancers in kidney and heart transplant recipients after the first squamous cell carcinoma. Transplantation, 81:1093-1100.
3. Fitzpatrick T B. The validity and practicality of sun-reactive skin types I through VI. Arch Dermatol (1988); 124(6):869.
4. Bouwes Bavinck J N, Hardie D R, Green A, et al. The risk of skin cancer in renal transplant recipients in Queensland, Australia. A follow-up study. Transplantation (1996); 61(5):715.
5. Bordea C, Wojnarowska F, Millard P R, et al. Skin cancers in renaltransplant recipients occur more frequently than previously recognized in a temperate climate. Transplantation (2004); 77(4):574.
6. Ramsay H M, Fryer A A, Hawley C M, et al. Non-melanoma skin cancer risk in the Queensland renal transplant population. Br J Dermatol (2002); 147(5):950.
7. Fuente M J, Sabat M, Roca J, et al. A prospective study of the incidence of skin cancer and its risk factors in a Spanish Mediterranean population of kidney transplant recipients. Br J Dermatol (2003); 149(6):1221.
8. Euvrard S, Kanitakis J, Claudy A. Skin cancers after organ transplantation. N Engl J Med (2003); 348(17): 1681.
9. Euvrard S, Kanitakis J, Pouteil-Noble C, et al. Comparative epidemiologic study of premalignant and malignant epithelial cutaneous lesions developing after kidney and heart transplantation. J Am Acad Dermatol (1995); 33(2 Pt 1):222.
10. Lindelof B, Sigurgeirsson B, Gabel H, Stern R S. Incidence of skin cancer in 5356 patients following organ transplantation. Br J Dermatol (2000); 143(3):513.
11. Fortina A B, Caforio A L, Piaserico S, et al. Skin cancer in heart transplant recipients: frequency and risk factor analysis. J Heart Lung Transplant (2000); 19(3):249.
12. Jensen P, Hansen S, Moller B, et al. Skin cancer in kidney and heart transplant recipients and different long-term immunosuppressive therapy regimens. J Am Acad Dermatol (1999); 40(2 Pt 1):177.
13. Ong C S, Keogh A M, Kossard S, et al. Skin cancer in Australian heart transplant recipients. J Am Acad Dermatol (1999); 40(1):27.
14. Birkeland S A, Storm H H, Lamm L U, et al. Cancer risk after renal transplantation in the Nordic countries, 1964-1986. Int J Cancer (1995); 60(2):183.
15. Abdel-Malek Z A, Kadekaro A C, Kavanagh R J et al. Melanoma prevention strategy based on using tetrapeptide α-MSH analogs that protect human melanocytes from UV-induced DNA damage and cytotoxicity. FASEB J. (2006); 20, E888-E896.

1-15. (canceled)

16. A method of treatment to reduce the incidence or rate of development of a non-melanoma skin cancer in an immune-compromised subject, which comprises the step of administering [Nle4, D-Phe7]-alpha-MSH to said subject.

17. The method of claim 16, wherein the subject is a human subject.

18. The method of claim 16, wherein the subject is an organ transplant patient.

19. The method of claim 16, wherein [Nle4, D-Phe7]-alpha-MSH is administered to the patient in association with immune suppressive medication.

20. The method of claim 16, wherein [Nle4, D-Phe7]-alpha-MSH is administered subcutaneously.

21. The method of claim 16, wherein the [Nle4, D-Phe7]-alpha-MSH is encapsulated in a delivery system selected from the group consisting of microcapsule, implant, and rod, in an amount of from 5% to 60% by weight.

22. The method of claim 16, wherein the non-melanoma skin cancer is selected from squamous cell carcinoma, basal cell carcinoma, and actinic keratosis.

23. The method of claim 16, wherein the subject has Fitzpatrick skin type I or II.

24. A method of treatment to reduce the incidence or rate of development of a non-melanoma skin cancer in an immune-compromised subject, which comprises the step of subcutaneously administering a sustained release implant comprising 20 mg [Nle4, D-Phe7]-alpha-MSH contained in a poly(D, L-lactide) implant core to said subject.

25. The method of claim 24, wherein the non-melanoma skin cancer is selected from squamous cell carcinoma, basal cell carcinoma, and actinic keratosis.

26. The method of claim 24, wherein the subject has Fitzpatrick skin type I or II.

27. A method of treatment to reduce the incidence or rate of development of a non-melanoma skin cancer in an immune-compromised subject, which comprises the step of administering to said subject an implant or rod comprising poly(lactide) or poly(lactide-co-glycolide) and an amount from 15% to 45% by weight of [Nle4, D-Phe7]-alpha-MSH.

28. The method of claim 27, wherein the non-melanoma skin cancer is selected from squamous cell carcinoma, basal cell carcinoma, and actinic keratosis.

29. The method of claim 27, wherein the subject has Fitzpatrick skin type I or II.


SEQUENCE LISTING (32 sequences)

SEQ ID NO: 1 - protein, 13 aa, Artificial Sequence
  Description of Artificial Sequence Synthetic peptide
SYSXQHFRWGKPV

SEQ ID NO: 2 - protein, 8 aa, Artificial Sequence
  Description of Artificial Sequence Synthetic peptide
XXXXXXXX

SEQ ID NO: 3 - protein, 13 aa, Artificial Sequence
  Description of Artificial Sequence Synthetic peptide
SYSXEHFRWKGPV

SEQ ID NO: 4 - protein, 13 aa, Artificial Sequence
  Description of Artificial Sequence Synthetic peptide
SYSXDHFRWKGPV

SEQ ID NO: 5 - protein, 10 aa, Artificial Sequence
  Description of Artificial Sequence Synthetic peptide
XEHFRWKGPV

SEQ ID NO: 6 - protein, 10 aa, Artificial Sequence
  Description of Artificial Sequence Synthetic peptide
XDHFRWKGPV

SEQ ID NO: 7 - protein, 7 aa, Artificial Sequence
  Description of Artificial Sequence Synthetic peptide
XDHFRWG

SEQ ID NO: 8 - protein, 7 aa, Artificial Sequence
  Description of Artificial Sequence Synthetic peptide
XEHFRWK

SEQ ID NO: 9 - protein, 7 aa, Artificial Sequence
  Description of Artificial Sequence Synthetic peptide
XDHFRWK

SEQ ID NO: 10 - protein, 7 aa, Artificial Sequence
  Description of Artificial Sequence Synthetic peptide
XEHFRWX

SEQ ID NO: 11 - protein, 7 aa, Artificial Sequence
  Description of Artificial Sequence Synthetic peptide
XDHFRWX

SEQ ID NO: 12 - protein, 7 aa, Artificial Sequence
  Description of Artificial Sequence Synthetic peptide
XEHFRWX

SEQ ID NO: 13 - protein, 7 aa, Artificial Sequence
  Description of Artificial Sequence Synthetic peptide
XDHFRWX

SEQ ID NO: 14 - protein, 7 aa, Artificial Sequence
  Description of Artificial Sequence Synthetic peptide
XEHFRWX

SEQ ID NO: 15 - protein, 7 aa, Artificial Sequence
  Description of Artificial Sequence Synthetic peptide
XEHFRWK

SEQ ID NO: 16 - protein, 7 aa, Artificial Sequence
  Description of Artificial Sequence Synthetic peptide
XDHFRWK

SEQ ID NO: 17 - protein, 10 aa, Artificial Sequence
  Description of Artificial Sequence Synthetic peptide
XEHFRWKGPV

SEQ ID NO: 18 - protein, 7 aa, Artificial Sequence
  Description of Artificial Sequence Synthetic peptide
XEHFRWK

SEQ ID NO: 19 - protein, 7 aa, Artificial Sequence
  Description of Artificial Sequence Synthetic peptide
XDHFRWK

SEQ ID NO: 20 - protein, 7 aa, Artificial Sequence
  Description of Artificial Sequence Synthetic peptide
XDHFRWX

SEQ ID NO: 21 - protein, 7 aa, Artificial Sequence
  Description of Artificial Sequence Synthetic peptide
XDHFRWX

SEQ ID NO: 22 - protein, 7 aa, Artificial Sequence
  Description of Artificial Sequence Synthetic peptide
XDHFRWX

SEQ ID NO: 23 - protein, 13 aa, Artificial Sequence
  Description of Artificial Sequence Synthetic peptide
SYSXDHFRWKGPV

SEQ ID NO: 24 - protein, 10 aa, Artificial Sequence
  Description of Artificial Sequence Synthetic peptide
SYSXDHFRWK

SEQ ID NO: 25 - protein, 9 aa, Artificial Sequence
  Description of Artificial Sequence Synthetic peptide
YSXDHFRWK

SEQ ID NO: 26 - protein, 8 aa, Artificial Sequence
  Description of Artificial Sequence Synthetic peptide
SXDHFRWK

SEQ ID NO: 27 - protein, 7 aa, Artificial Sequence
  Description of Artificial Sequence Synthetic peptide
XDHFRWK

SEQ ID NO: 28 - protein, 8 aa, Artificial Sequence
  Description of Artificial Sequence Synthetic peptide
XDHFRWKG

SEQ ID NO: 29 - protein, 9 aa, Artificial Sequence
  Description of Artificial Sequence Synthetic peptide
XDHFRWKGP

SEQ ID NO: 30 - protein, 10 aa, Artificial Sequence
  Description of Artificial Sequence Synthetic peptide
XDHFRWKGPV

SEQ ID NO: 31 - protein, 11 aa, Artificial Sequence
  Description of Artificial Sequence Synthetic peptide
SXDHFRWKGPV

SEQ ID NO: 32 - protein, 4 aa, Artificial Sequence
  Description of Artificial Sequence Synthetic peptide
HFRW